(19) United States
(12) Patent Application Publication
(10) Pub. No.: US 2006/0257347 A1
(43) Pub. Date: Nov. 16, 2006

(54) Self-preservation system

(76) Inventors: Joo Woun Kim, Mississauga (CA); Michael D. Hooper, Oakville (CA)

Correspondence Address: The Halvorson Law Firm;Ste 130, 1757 E. Baseline Rd., Gilbert, AZ, 85233, US

(21) Appl. No.: 11/128,534

(22) Filed: May 13, 2005

(51) Int. Cl. A61K 8/73

(52) U.S. Cl. 424/70.13; 424/70.31; 424/70.22; 424/70.28

(57) ABSTRACT

Babassuamadopropalkonium chloride is quaternary ammonium compounds obtained from natural babassu oil that contains a high level of lauric acid, and it has been used as mild conditioner. We found that babassuamadopropalkonium chloride has strong and broad-spectrum antimicrobial activity especially with bisabolol. Bisabolol is the active component of Chamomile, one of the most used medical plants, and has been used as anti-irritant. We found that there is a synergistic effect of bisabolol on hinokitiol, when combined with ethylhexylglycerin or caprylyl glycol. This synergistic effect of composition of bisabolol and ethylhexylglycerin can reduce the required amount of hinokitiol, which if used in high amount can be unstable by more than 100 times. Self-preserving personal care cleansing compositions that include bisabolol, alkyl or alkylamidopropyl betaines, amphoteric surfactants, acyl sarcosinates and optionally fatty acids and(or) acyl lactylate and(or) acyl glucoside and(or) babassuamadopropalkonium chloride and colloidal silver have also broad-spectrum antimicrobial activity.

This invention relates to antimicrobial compositions that can be used as self-preservation system for personal care products.

### BACKGROUND AND PRIOR ART

Natural personal care products and remedies are popular among health-conscious consumers today. Many people prefer to enhance their appearance and health with vitamins and other nutrients in a “natural” way from naturally occurring sources. “Natural” products including natural vitamins, and especially preservatives, are now in high demand. These are components that are derived from a product in its natural state without supplementation.

Previously, chemical preservatives have been used to prevent bacterial or fungal growth in personal care products. Typical chemical preservatives include, in particular, parabens or formaldehyde donors. These preservatives present the drawback of causing intolerance, such as irritations and/or allergies particularly on sensitive skins.

Many natural antibacterial components are weak or have their antibacterial effect masked by other constituents of the composition. For example, U.S. Pat. Nos. 5,260,053 and 5,256,405 are prepared deodorants that contain active antibacterial constituents consisting of 1.00-6.00 wt % lichen Extract, 0.10 wt % -3.00 wt % coriander oil, and 0.10 wt % -0.60 wt % glyceryl monolaurate. These compositions have only a weak antibacterial effect and do not inhibit the growth of fungi. These cannot be applied as self-preservatives for preserving personal care products.

What is needed are personal care products that deliver natural vitamins, nutrients and other beneficial products to the skin and hair and clean them without using chemical preservatives and their adverse side-effects.

The novel features that are considered characteristics of the invention are set forth with particularity in the appended claims. The invention itself, however, both as to its structure and its operation together with the additional object and advantages. thereof, will best be understood from the following description of the preferred embodiment of the present invention when read in conjunction with any accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or a phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material, or acts that are described in the preferred embodiments, but in addition, include any and all structures, materials, or acts that perform the claimed function, along with any and all known or later-developed equivalent structures, materials, or acts for performing the claimed function.

### SUMMARY OF THE INVENTION

It is an object of the present invention to provide personal care products that include natural and(or) nature-originated antimicrobial/antifungal compositions.

It is another object of the present invention to provide personal care products that include natural and(or) nature-originated antimicrobial/antifungal compositions that comprise simple type emulsions, conditioners, and gels, comprising between 0.10-0.30 wt % bisabolol and between 0.10-0.50 wt % babassuamadopropalkonium chloride. These compositions have a surprising and unexpected synergistic effect against microbes and fungi.

It is yet another object of the present invention to provide personal care products that are comprised of formulas which consist high content of vitamins, proteins, lecithin, polysorbates, and natural and(or) nature-originated antimicrobial/antifungal compositions that are comprised of between 0.10-0.30 wt % bisabolol, between 0.30-0.70 wt % ethylhexylglycerin, caprylyl glycol, and between 0.00-0.50 wt % babassuanadopropalkonium chloride and(or) between 0.05-0.50 wt % hinokitiol which are used as natural anti-microbial.

It is another object of the present invention to provide self-preserving personal care cleansing compositions that comprise of between 15.00-50.00 wt % alkyl or alkylamidopropyl betaines, between 5.00-20.00 wt % amphoteric surfactants and(or) between 0.00-20.00 wt % acyl sarcosinate and(or) between 0.00-20.00 wt % alkyl glucoside as main surfactants and as self-preservation compositions, between 0.00-10.00 wt % acyl lactylate and(or) between 0.00-5.00 wt % fatty acid and(or) 0.00-2.00 wt % babassuamadopropalkonium chloride as viscosity builder, between 20.00-50.00 ppm colloidal silver, which when combined with between 0.00-0.50 wt % of bisabolol functions as a booster of antimicrobial activity of main surfactants.

### DESCRIPTION OF THE PREFERRED EMBODIMENT

The present invention is a natural and(or) nature-originated composition of preservation for personal care products that exhibit broad range antimicrobial and antifungal activity.

This natural and(or) nature-originated composition of preservation is compatible with nonionic surfactants, the most type of polymers, cationic surfactants, amphoteric surfactants, and alkyl or alkylamidopropyl betaines.

The formula for Example 1, Table 1, is a simple anionic polymer gel that demonstrates the efficacy of the Bisabolol and babassuamadopropalkonium chloride composition against a wide range of microorganisms, see Table 2. As illustrated in Table 2, bisabolol and babassuamadopropalkonium chloride composition was effective against the five most common test microorganisms: Escherichia coli, Pseudomonas aeruginosa, Staphylococcus aureus, Candida albicans, and Aspergillus niger. Even after 4 weeks, these microorganisms did not recolonize their samples. This result also demonstrates that the efficacy of this bisabolol and babassuamadopropalkonium chloride composition is not changed by anionic polymers.

TABLE 1
Test Composition

| Compound 1 | Wt % |
|---|---|
| Carbomer Ultrez 10 | 0.30 |
| Triethanolamine | 0.30 |
| Bisabolol | 0.20 |
| Babassuamadopropalkonium Chloride | 0.30 |
| Water | to 100 |

TABLE 2
Challenge test results for Table 1 formula (USP method, 5 hr to 4 weeks
recovery)

| | Initial |
|---|---|
| Microorganism | Concentration (CFU/ml) |
| Escherichia coli | 3.1 * 10⁶ |
| (E. coli. ATCC No. 8739) | |
| Pseudomonas aeruginosa | 3.4 * 10⁶ |
| (P. aeruginosa ATCC No. 9027) | |
| Staphylococcus aureus | 2.8 * 10⁶ |
| (S. aureus ATCC No. 6538) | |
| Candida albicans | 7.4 * 10⁶ |
| (C. albicans ATCC No. 10231) | |
| Aspergillus niger | 5.3 * 10⁵ |
| (A. niger ATCC No. 16404) | |

| | E. coli | P. aeruginosa | S. aureus | C. albicans | A. niger |
|---|---|---|---|---|---|
| 5 hr | 0 | 0 | 0 | 0 | <10 |
| 24 hr | 0 | 0 | 0 | 0 | 0 |
| 3 days | 0 | 0 | 0 | 0 | 0 |
| 1 week | 0 | 0 | 0 | 0 | 0 |
| 2 weeks | 0 | 0 | 0 | 0 | 0 |
| 4 weeks | 0 | 0 | 0 | 0 | 0 |

The formulation for Example 2, Table 3, demonstrates that the bisabolol, babassuamadopropalkonium chloride, and ethylhexylglycerin compositions which are in the formulation that includes 5.00 wt % of polysorbates is totally effective against the five test microorganisms. But less than 0.30 wt % of babassuamadopropalkonium chloride, even with 0.60 wt % of ethylhexylglycerin, is not effective against P. aeruginosa.

Thus, formulas that contain small amount of protein, polysorbates, and any cosmetic actives can be preserved by adding only between 0.00 and 0.20 wt % bisabolol and between 0.20 and 0.30 wt % babassuamadopropalkonium chloride. However, formulas that contain high amounts of protein and polysorbates can be preserved by adding between 0.20 and 0.50 wt % bisabolol, between 0.30 and 0.50 wt % babassuamadopropalkonium chloride, and between 0.30 and 1.00 wt % ethylhexylglycerin.

TABLE 3

| Compound 2 | wt % |
|---|---|
| Hydroxyethylcellulose | 0.30 |
| Polysorbate 20 | 5.00 |
| Bisabolol | 0.20 |
| Ethylhexylglycerin | 0.60 |
| Babassuamadopropalkonium Chloride | 0.10-0.3 |
| Water | to 100% |

TABLE 4
Challenge test results for Table 3 formula (USP method, 28 days
recovery).

| | Initial |
|---|---|
| Microorganism | Concentration (CFU/ml) |
| Escherichia coli | 3.6 * 10⁶ |
| (E. coli. ATCC No. 8739) | |
| Pseudomonas aeruginosa | 4.4 * 10⁶ |
| (P. aeruginosa ATCC No. 9027) | |
| Staphylococcus aureus | 3.9 * 10⁶ |
| (S. aureus ATCC No. 6538) | |
| Candida albicans | 5.5 * 10⁶ |
| (C. albicans ATCC No. 10231) | |
| Aspergillus niger | 5.7 * 10⁵ |
| (A. niger ATCC No. 16404) | |

| | E. | | | | |
|---|---|---|---|---|---|
| | coli | P. aeruginosa | S. aureus | C. albicans | A. niger |
| BAC 0.10% | 0 | 5.0 * 10⁶ | 0 | 0 | <10 |
| BAC 0.30% | 0 | 0 | | 0 | 0 |

BAC = babassuamadopropalkonium Chloride

The formulation for Example 3, Table 5, which is a sunscreen composition that does not include proteins, polysorbates, and another cosmetic actives, further demonstrates that different components in the formulations can mask the antibacterial properties of babassuamadopropalkonium chloride and bisabolol. However, this example also demonstrates the synergistic effect of bisabolol on the antimicrobial activity of babassuamadopropalkonium chloride. Table 6 clearly demonstrates that a sunscreen formulation containing 0.30 wt % of babassuamadopropalkonium chloride only decreased the P. aereginosa concentration by approximately half. But a composition that contained 0.20 wt % bisabolol and 0.30 wt % babassuamadopropalkonium chloride was fully effective against P. aereginosa, contrary to the results in Table 4, where the test composition of Compound 1, which had the same babassuamadopropalkonium Chloride concentrations, was essentially ineffective against P. aereginosa. And even only 0.10 wt % babassuamadopropalkonium chloride with 0.20 wt % bisabolol is shown to be totally effective against the five test micro-organisms. This result demonstrates the synergistic effect of bisabolol on the antimicrobial activity of babassuamadopropalkonium chloride.

TABLE 5
Sunscreen

| Compound 4 | wt % |
|---|---|
| Xanthan Gum | 0.40 |
| Cetyl Alcohol | 1.00 |
| Glycerin | 3.00 |
| Dimethicone | 2.00 |
| Isopropylpalmitate | 3.00 |
| Emulsifying Wax NF | 6.00 |
| Octyl Methoxycinnamate | 7.50 |
| Benzophenone-3 | 6.00 |
| Octocylene | 10.00 |
| Octyl Salicylate | 5.00 |
| Babassuamadopropalkonium Chloride | 0.10-0.30 |
| Bisabolol | 0.00-0.20 |
| Water | to 100 |

TABLE 6
Challenge test results for Table 5 formula (USP method, 28 days
recovery)

| | Initial |
|---|---|
| Microorganism | Concentration (CFU/ml) |
| Escherichia coli | 3.1 * 10⁶ |
| (E. coli. ATCC No. 8739) | |
| Pseudomonas aeruginosa | 3.5 * 10⁶ |
| (P. aeruginosa ATCC No. 9027) | |
| Staphylococcus aureus | 3.5 * 10⁶ |
| (S. aureus ATCC No. 6538) | |
| Candida albicans | 4.7 * 10⁶ |
| (C. albicans ATCC No. 10231) | |
| Aspergillus niger | 6.8 * 10⁵ |
| (A. niger ATCC No. 16404) | |

| | E. | P. | | | |
|---|---|---|---|---|---|
| | coil | aeruginosa | S. aureus | C. albicans | A. niger |
| BAC 0.30% + | 0 | 3.0 * 10³ | 0 | 0 | 0 |
| AB 0.00% | | | | | |
| BAC 0.30% + | 0 | 0 | 0 | 0 | 0 |
| AB 0.20% | | | | | |
| BAC 0.10% + | 0 | 0 | 0 | 0 | 0 |
| AB 0.20% | | | | | |

BAC = babassuamadopropalkonium Chloride
AB = Bisabolol

The formulation for Example 4, Table 7, which is a skin moisturizing cream, containing high content protein and other cosmetic active ingredients, demonstrates the antimicrobial boosting effect of the ethylhexylglycerin on the composition of 0.20 wt % of bisabolol and 0.30 wt % of babassuamadopropalkonium chloride, which was also proven effective for the compositions in Example 2. In these cases, Table 7 demonstrates that the addition of a modest amount, 0.60 wt. % of ethylhexylglycerin or caprylyl glycol can restore full antimicrobial efficacy.

TABLE 7
Skin Moisturizing Cream

| Compound 3 | wt % |
|---|---|
| Allantoin | 0.25 |
| Hydroxyethylcellulose | 0.30 |
| Glycerin | 3.00 |
| Panthenol dl | 2.00 |
| Emulsifying Wax NF | 6.00 |
| Sunflower Oil | 9.00 |
| Sweet almond oil | 1.00 |
| Dimethicone | 2.00 |
| Cetyl Alcohol | 3.00 |
| Stearyl Alcohol | 2.00 |
| Tocopheryl (Vitamin E) Acetate | 2.00 |
| Lecithin Unbleached, F.C.C. | 0.50 |
| Ascorbyl Palmitate | 2.00 |
| Tocopherol | 0.20 |
| Hydrolyzed Soy Protein | 2.00 |
| Babassuamadopropalkonium Chloride | 0.30 |
| Bisabolol | 0.20 |
| Ethylhexylglycerin | 0.00-0.60 |
| Caprylyl glycol | 0.00-0.60 |
| Water | to 100 |

TABLE 8
Challenge test results for Table 7 formula (USP method, 28 days
recovery).

| | Initial |
|---|---|
| Microorganism | Concentration (CFU/ml) |
| Escherichia coli | 3.1 * 10⁶ |
| (E. coli. ATCC No. 8739) | |
| Pseudomonas aeruginosa | 3.5 * 10⁶ |
| (P. aeruginosa ATCC No. 9027) | |
| Staphylococcus aureus | 3.5 * 10⁶ |
| (S. aureus ATCC No. 6538) | |
| Candida albicans | 4.7 * 10⁶ |
| (C. albicans ATCC No. 10231) | |
| Aspergillus niger | 6.8 * 10⁵ |
| (A. niger ATCC No. 16404) | |

| | E. | | | C. | |
|---|---|---|---|---|---|
| | coli | P. aeruginosa | S. aureus | albicans | A. niger |
| EHG 0.00% | 0 | 0 | 0 | 7.2 * 10⁵ | 2.8 * 10³ |
| EHG 0.60% | 0 | 0 | 0 | 0 | 0 |
| CPG 0.60% | 0 | 0 | 0 | 0 | 0 |

EHG = Ethylhexylglycerin
CPG = Caprylyl glycol

The formulation for Example 5, Table 9, which is a moisturizing lotion, demonstrates that as little as 0.05 wt. % hinokitiol can replace 0.30 wt % babassuamadopropalkonium chloride in some formulations that contain 0.20 wt % bisabolol, 0.60 wt % caprylyl glycol, and the 0.30 wt % babassuamadopropalkonium chloride and can still remain effective against a broad range of micro-organisms, Table 10.

TABLE 9
Moisturizing Lotion

| Compound 5 | wt % |
|---|---|
| l-Arginine | 2.00 |
| Lactic Acid | 1.40 |
| Cyclomethicone | 3.00 |
| Olive Oil | 6.00 |
| Sunflowseed Oil | 2.50 |
| Stearyl Alcohol | 2.50 |
| Cetyl Alcohol | 2.50 |
| Steareth-20 | 1.30 |
| Steareth-2 | 2.00 |
| Hyaluronic Acid | 0.10 |
| Glycerin | 3.00 |
| Sodium PCA | 4.00 |
| Tocopherol | 0.20 |
| Colostrum | 0.50 |
| Alpha Lipids | 0.50 |
| Lactoferrin | 0.50 |
| Caprylyl glycol | 0.60 |
| Bisabolol | 0.20 |
| Babassuamadopropalkonium Chloride | 0.00-0.30 |
| Hinokitiol | 0.00-0.05 |
| Water | to 100 |

TABLE 10
Challenge test results for Table 9 formula (USP method, 28 days
recovery)

| | Initial |
|---|---|
| Microorganism | Concentration (CFU/ml) |
| Escherichia coli | 4.2 * 10⁶ |
| (E. coli. ATCC No. 8739) | |
| Pseudomonas aeruginosa | 5.1 * 10⁶ |
| (P. aeruginosa ATCC No. 9027) | |
| Staphylococcus aureus | 4.6 * 10⁶ |
| (S. aureus ATCC No. 6538) | |
| Candida albicans | 4.6 * 10⁶ |
| (C. albicans ATCC No. 10231) | |
| Aspergillus niger | 5.9 * 10⁵ |
| (A. niger ATCC No. 16404) | |

| | E. | P. | | C. | |
|---|---|---|---|---|---|
| | coli | aeruginosa | S. aureus | albicans | A. niger |
| BAC 0.00% + | 0 | 0 | 0 | 0 | 4.2 * 10⁴ |
| HK 0.00% | | | | | |
| BAC 0.30% + | 0 | 0 | 0 | 0 | 0 |
| HK 0.00% | | | | | |
| BAC 0.00% + | 0 | 0 | 0 | 0 | 0 |
| HK 0.05% | | | | | |

BAC = babassuamadopropalkonium Chloride
HK = Hinokitiol

The synergistic effect of bisabolol and ethylhexylglycerin on the antimicrobial effect of the hinokitiol is demonstrated in Example 6. The MICs (Minimum Inhibitory Concentrations) of hinokitiol for the five test micro-organisms are known to be

| | |
|---|---|
| Candida albicans (ATCC No. 10231) | 12.5 mg/ml |
| Aspergillus niger (ATCC No. 16404) | 40.0 mg/ml |
| Escherichia coli (ATCC No. 8739) | 40.0 mg/ml |
| Pseudomonas aeruginosa (ATCC No. 9027) | 50.0 mg/ml |
| Staphylococcus aureus (ATCC No. 6538) | 40.0 mg/ml |

However, the following composition demonstrates that the MICs of hinokitiol are greatly reduced by the addition of bisabolol and ethylhexylglycerin.

| Compound | wt % |
|---|---|
| Bisabolol | 24.70 |
| Ethylhexylglycerin | 74.00 |
| Hinokitiol | 1.30 |
| Candida albicans (ATCC No. 10231) | lower than 0.062 mg/ml |
| Aspergillus niger (ATCC No. 16404) | lower than 0.062 mg/ml |
| Escherichia coli (ATCC No. 8739) | lower than 0.124 mg/ml |
| Pseudomonas aeruginosa (ATCC No. 9027) | lower than 0.496 mg/ml |
| Staphylococcus aureus (ATCC No. 6538) | lower than 0.062 mg/ml |

The shampoo formulas of Example 7, Table 11, demonstrate that the personal care cleansing products that are comprised of alkyl or alkylamidopropyl betaines, acyl sarcosinate, acyl lactylate, and bisabolol can be self-preserved. But this composition is not sufficient to eradicate the A. niger. Further babassuamadopropalkonium chloride does not increase the antifungal effect of this composition.

TABLE 11

| Compound | wt % |
|---|---|
| Cocamidopropyl Betain | 34.00 |
| Sodium Cocoyl Sarcosinate | 12.50 |
| Sodium Lauroyl Lactylate | 3.40 |
| Bisabolol | 0.10 |
| Sodium Hydroxide | make pH to 5.5 to 6.5 |
| Babassuamadopropalkonium Chloride | 0.00-0.30 |
| Soyamidopropalkonium Chloride | 0.30 |
| Water | to 100% |

TABLE 12
Challenge test results for Table 11 formula (USP method, 28 days
recovery)

| Microorganism | Initial Concentration (CFU/ml) |
|---|---|
| Escherichia coli | 3.1 * 10⁵ |
| (E. coli. ATCC No. 8739) | |
| Pseudomonas aeruginosa | 3.4 * 10⁵ |
| (P. aeruginosa ATCC No. 9027) | |
| Staphylococcus aureus | 2.8 * 10⁵ |
| (S. aureus ATCC No. 6538) | |
| Candida albicans | 7.4 * 10⁵ |
| (C. albicans ATCC No. 10231) | |
| Aspergillus niger | 5.8 * 10⁴ |
| (A. niger ATCC No. 16404) | |

| | E. | P. | | | |
|---|---|---|---|---|---|
| | coli | aeruginosa | S. aureus | C. albicans | A. niger |
| BAC 0.00% | 0 | 0 | 0 | 0 | 6.10 * 10³ |
| BAC 0.10% | 0 | 0 | 0 | 0 | 1.20 * 10⁴ |
| BAC 0.30% | 0 | 0 | 0 | 0 | 3.20 * 10³ |

BAC = Babassuamadopropalkonium Chloride

Compositions of Example 8 (Table 13) containing alkyl or alkylamidopropyl betaines, amphoteric surfactants, colloidal silver, acyl sarcosinate, bisabolol, and babassuamadopropalkonium chloride have full antimicrobial effect against the test microorganisms (Table 14).

TABLE 13

| Compound | wt % |
|---|---|
| Cocamidopropyl Betain | 30.00 |
| Disodium Cocoamphodiacetate | 14.50 |
| Sodium Cocoyl Sarcosinate | 7.00 |
| Colloidal Silver | 20 ppm |
| Bisabolol | 0.10 |
| Babassuamadopropalkonium Chloride | 0.60 |
| Fragrance | 0.50 |
| Water | to 100% |

TABLE 14
Challenge test results for Table 13 formula (USP method, 28 days
recovery)

| | Initial |
|---|---|
| Microorganism | concentration (CFU/ml) |
| Escherichia coli | 4.0 * 10⁶ |
| (E. coli. ATCC No. 8739) | |
| Pseudomonas aeruginosa | 2.7 * 10⁵ |
| (P. aeruginosa ATCC No. 9027) | |
| Staphylococcus aureus | 6.6 * 10⁶ |
| (S. aureus ATCC No. 6538) | |
| Candida albicans | 7.2 * 10⁶ |
| (C. albicans ATCC No. 10231) | |
| Aspergillus niger | 2.3 * 10⁵ |
| (A. niger ATCC No. 16404) | |

| | E. coli | P. aeruginosa | S. aureus | C. albicans | A. niger |
|---|---|---|---|---|---|
| 1 week | 0 | 0 | 0 | 0 | 0 |
| 2 weeks | 0 | 0 | 0 | 0 | 0 |
| 3 weeks | 0 | 0 | 0 | 0 | 0 |
| 4 weeks | 0 | 0 | 0 | 0 | 0 |
| 5 weeks | 0 | 0 | 0 | 0 | 0 |

The formulas of Example 9, Table 15, demonstrate that even relatively large amounts of babassuamadopropalkonium chloride, in the absence of colloidal silver, are ineffective against A. niger. However, even a relatively small amount, less than 20 ppm, of colloidal silver is sufficient to eradicate the A. niger micro-organism. Note that in the absence of bisabolol, amphoteric surfactant, colloidal silver is relatively ineffective against A. niger. In order to eradicate A. niger higher than 100.00 ppm of colloidal silver is required.

TABLE 15
Shampoo

| Compound | wt % |
|---|---|
| Cocamidopropyl Betain | 30.00 |
| Disodium Cocoamphodiacetate | 13.00 |
| Sodium Cocoyl Sarcosinate | 7.00 |
| Colloidal Silver | 0.00-20.00 ppm |
| Bisabolol | 0.10 |
| Babassuamadopropalkonium Chloride | 0.00-0.90 |
| Citric acid | make pH 5.0 to 7.0 |
| Fragrance | 0.40 |
| Water | to 100% |

TABLE 16
Challenge test results for Table 15 formula (USP method, 28 days
recovery)

| | Initial |
|---|---|
| Microorganism | concentration (CFU/ml) |
| Escherichia coli | 4.0 * 10⁶ |
| (E. coli. ATCC No. 8739) | |
| Pseudomonas aeruginosa | 2.7 * 10⁵ |
| (P. aeruginosa ATCC No. 9027) | |
| Staphylococcus aureus | 6.6 * 10⁶ |
| (S. aureus ATCC No. 6538) | |
| Candida albicans | 7.2 * 10⁶ |
| (C. albicans ATCC No. 10231) | |
| Aspergillus niger | 2.3 * 10⁵ |
| (A. niger ATCC No. 16404) | |

| | E. | P. | S. | | |
|---|---|---|---|---|---|
| | coli | aeruginosa | aureus | C. albicans | A. niger |
| BAC 0.00%/ | 0 | 0 | 0 | 0 | 4.4 * 10² |
| CS 0.00 ppm | | | | | |
| BAC 0.60%/ | 0 | 0 | 0 | 0 | 2.3 * 10² |
| CS 0.00 ppm | | | | | |
| BAC 0.90%/ | 0 | 0 | 0 | 0 | 1.5 * 10² |
| CS 0.00 ppm | | | | | |
| BAC 0.00%/ | 0 | 0 | 0 | 0 | 3.1 * 10² |
| CS 5.00 ppm | | | | | |
| BAC 0.00%/ | 0 | 0 | 0 | 0 | 0 |
| CS 20.00 ppm | | | | | |
| BAC 0.30%/ | 0 | 0 | 0 | 0 | 0 |
| CS 20.00 ppm | | | | | |
| BAC 0.60%/ | 0 | 0 | 0 | 0 | 0 |
| CS 20.00 ppm | | | | | |

BAC = Babassuamadopropalkonium Chloride
CS = Colloidal Silver

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A natural personal care product preserving composition comprising between 0.01 and 0.50 wt % bisabolol.

2. The natural personal care product preserving composition according to claim 1 further comprising between 0.01 and 0.50 wt % babassuamadopropalkonium chloride.

3. The natural personal care product preserving composition according to claim 2 further comprising between 0.01 and 1.00 wt % ethylhexylglycerin or caprylyl glycol.

4. The natural personal care product preserving composition according to claim 1 further comprising between 0.01 and 1.00 wt % ethylhexylglycerin and between 0.001 and 0.50 wt % hinokitiol.

5. The natural personal care product preserving composition according to claim 1 further comprising between 15.00 and 50.00 wt % alkyl or alkylamidopropyl betaines, between 5.00 and 20.00 wt % amphoteric surfactants, and(or) between 0.00 and 20.00 wt % acyl sarcosinates as main surfactants and anti-microbial agents.

6. The natural personal care product preserving composition according to claim 5 further comprising between 0.00 and 10.00 wt % acyl lactylates and(or) between 0.00 and 20.00 wt % alkyl glucoside and(or) between 0.00 and 5.00 wt % fatty acid and(or) 0.00 and 2.00 wt % babassuamadopropalkonium chloride as viscosity builders.

7. The natural personal care product preserving composition according to claim 5 further comprising between 20.00 and 50.00 ppm colloidal silver as antifungal activity booster.
